(19) United States
(12) Patent Application Publication
(10) Pub. No.: US 2006/0106106 A1
(43) Pub. Date: May 18, 2006

(54) Beta-hydroxyphenylalkylamines and their use for treating glaucoma

(76) Inventors: Richard A. Glennon, Richmond, VA (US); Mark R. Hellberg, Arlington, TX (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/526,076

(22) PCT Filed: Sep. 22, 2003
(86) PCT No.: PCT/US03/29818

§ 371 Date: Oct. 24, 2005

Related U.S. Application Data
(60) Provisional application No. 60/412,787, filed on Sep. 24, 2002.

(51) Int. Cl. A61K 31/205; A61K 31/138

(52) U.S. Cl. 514/554; 514/651

(57) ABSTRACT

β-hydroxyphenylalkylamines and their use for lowering and controlling ocular hypertension and treating glaucoma are disclosed.

### BACKGROUND OF THE INVENTION

The present invention relates to various β-hydroxyphenylalkylamines. These compounds, some of which are novel, are useful for lowering and controlling normal or elevated intraocular pressure (IOP) and for treating glaucoma.

The disease state referred to as glaucoma is characterized by a permanent loss of visual function due to irreversible damage to the optic nerve. The several morphologically or functionally distinct types of glaucoma are typically characterized by elevated IOP, which is considered to be causally related to the pathological course of the disease. Ocular hypertension is a condition wherein intraocular pressure is elevated, but no apparent loss of visual function has occurred; such patients are considered to be at a high risk for the eventual development of the visual loss associated with glaucoma. If glaucoma or ocular hypertension is detected early and treated promptly with medications that effectively reduce elevated intraocular pressure, loss of visual function or its progressive deterioration can generally be ameliorated. Drug therapies that have proven to be effective for the reduction of intraocular pressure include both agents that decrease aqueous humor production and agents that increase the outflow facility. Such therapies are in general administered by one of two possible routes, topically (direct application to the eye) or orally.

There are some individuals who do not respond well when treated with certain existing glaucoma therapies. There is, therefore, a need for other topical therapeutic agents that control IOP.

Serotonergic 5-HT_{1A} agonists have been reported as being neuroprotective in animal models and many of these agents have been evaluated for the treatment of acute stroke among other indications. This class of compounds has been mentioned for the treatment of glaucoma (lowering and controlling IOP), see e.g., WO 98/18458 (DeSantis, et al.) and EP 0771563A2 (Mano, et al.). Osborne, et al. (Ophthalmologica, Vol. 210:308-314, 1996) teach that 8-hydroxydipropylaminotetralin (8-OH-DPAT) (a 5-HT_{1A} agonist) reduces IOP in rabbits. Wang, et al. (Current Eye Research, Vol. 16(8):769-775, August 1997, and IVOS, Vol. 39(4), S488, March, 1998) indicate that 5-methylurapidil, an α_{1A} antagonist and 5-HT_{1A} agonist lowers IOP in the monkey, but due to its α_{1A} receptor activity. Also, 5-HT_{1A} antagonists are disclosed as being useful for the treatment of glaucoma (elevated IOP) (e.g., WO 92/0338, McLees). Furthermore, DeSai, et al. (WO 97/35579) and Macor, et al. (U.S. Pat. No. 5,578,612) relate to the use of 5-HT₁ and 5-HT₁₋ₗᵢₖₑ agonists for the treatment of glaucoma (elevated IOP). These anti-migraine compounds are 5-HT_{1B,D,E,F} agonists, e.g., sumatriptan and naratriptan and related compounds.

It has been found that serotonergic compounds which possess agonist activity at 5-HT₂ receptors effectively lower and control normal and elevated IOP and are useful for treating glaucoma, see pending application, U.S. Ser. No. 09/787,332 (WO 00/16761), incorporated herein by reference. Compounds that act as agonists at 5-HT₂ receptors are well known and have shown a variety of utilities, primarily for disorders or conditions associated with the central nervous system (CNS). U.S. Pat. No. 5,494,928 relates to certain 2-(indol-1-yl)-ethylamine derivatives that are 5-HT_{2C} agonists for the treatment of obsessive compulsive disorder and other CNS derived personality disorders. U.S. Pat. No. 5,571,833 relates to tryptamine derivatives that are 5-HT₂ agonists for the treatment of portal hypertension and migraine. U.S. Pat. No. 5,874,477 relates to a method for treating malaria using 5-HT_{2A/2C} agonists. U.S. Pat. No. 5,902,815 relates to the use of 5-HT_{2A} agonists to prevent adverse effects of NMDA receptor hypo-function. WO 98/31354 relates to 5-HT_{2B} agonists for the treatment of depression and other CNS conditions. WO 00/12475 relates to indoline derivatives and WO 00/12510 and WO 00/44753 relate to certain indole derivatives as 5-HT_{2B} and 5-HT_{2C} receptor agonists for the treatment of a variety of disorders of the central nervous system, but especially for the treatment of obesity. WO 00/35922 relates to certain pyrazino[1,2-a]quinoxaline derivatives as 5-HT_{2C} agonists for the treatment of obsessive compulsive disorder, depression, eating disorders, and other disorders involving the CNS. WO 00/77002 and WO 00/77010 relate to certain substituted tetracyclic pyrido[4,3-b]indoles as 5-HT_{2C} agonists with utility for the treatment of central nervous system disorders including obesity, anxiety, depression, sleep disorders, cephalic pain, and social phobias among others. Agonist response at the 5-HT_{2A} receptor is reported to be the primary activity responsible for hallucinogenic activity, with some lesser involvement of the 5-HT_{2A} receptor possible [Psychopharmacology, Vol. 121:357, 1995].

Certain β-hydroxy or alkoxy 2,5-methoxyphenylalkylamines have been prepared. β-Hydroxy (2,5-dimethoxyphenyl)propylamine has been prepared as an intermediate in the synthesis of radio-labeled methoxamine, an alpha adrenergic agonist [DeMarinis, et al., J. Labelled Compound Radiopharm., Vol. 9(2):267-70, 1982]. β-Hydroxy (2,5-dimethoxyphenyl)phenethyl methylamine has been prepared and used as a synthetic intermediate in the synthesis of hypolipidemic and hypoglycemic agents [Barfknecht, et al., Journal of Medicinal Chemistry, Vol. 17(3):308-312, 1974]. Other compounds have been prepared and studied for their CNS activity. β-hydroxy-2,5-dimethoxy amphetamine analogs were prepared and suggested to have hallucinogenic and/or sympathomimetic activity [Beng, et al., Journal of Medicinal Chemistry, Vol. 13(5):1022, 1970]. A series of β-methoxy phenyethylamine analogs have been prepared and evaluated for their psychotomimetic activity [Lemaire, et al., Journal Pharm. Pharmacol., Vol. 37(8):575-577, 1985]. A similar series of 4-substituted β-methoxy 2,5-dimethoxyphenyethylamine analogs has been prepared [Torres, et al., Synthetic Communications, Vol. 25(8):1239-1247, 1995] and evaluated for serotonergic and adrenergic activity [Torres, et al., Gen. Pharmac., Vol. 31(1):51-54, 1998]. The biological activity data derived from studies with many of these compounds has been used to generate structure activity relationships for hallucinogenic phenalkylamines [Beuerle, et al., Quantitative Structure Activity Relationships, Vol. 16(6):447-458, 1997 and Clare, B. W., J. Med. Chem., Vol. 41(20):3845-3856, 1998].

All the patents and publications mentioned above and throughout are herein incorporated in their entirety by reference.

Accordingly, there is a need to provide new compounds which avoid the disadvantages described above and which provide increased chemical stability and a desired length of therapeutic activity, for instance, in decreasing intraocular pressure and treating glaucoma. In addition, there is a need to provide improved method of lowering and/or controlling elevated intraocular pressure (IPO).

### SUMMARY OF THE PRESENT INVENTION

A feature of the present invention is to provide novel compounds which are 5-HT₂ agonists.

Another feature of the present invention is to provide compounds which have increased chemical stability and which are useful in lowering and controlling normal or elevated intraocular pressure and/or treating glaucoma.

Another feature of the present invention is to provide compounds which have less CNS activity than other known 5-HT₂ agonists.

Another feature of the present invention is to provide compounds which provide a desired level of therapeutic activity in lowering and controlling normal or elevated intraocular pressure and/or treating glaucoma.

To achieve these and other advantages, and in accordance with the purposes of the present invention, as embodied and broadly described herein, the present invention relates to a compound having the Formula I: 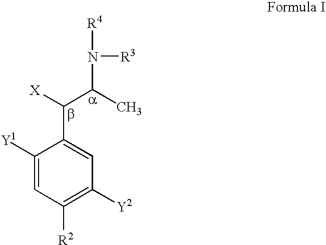
Wherein:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹ and Y² are not OH;
R¹=C₁₋₃ alkyl;
R²=C₁₋₃ alkyl, Cl, Br, I CF₃, or OR¹;
R³, R⁴=H, C₁₋₃ alkyl;
R⁵=C₁₋₆ alkyl; and
R⁶=H, C₁₋₆ alkyl.
   Preferred compounds for lowering and maintaining IOP or treating glaucoma include compounds wherein:
R¹=methyl;
R²=Br, C₁₋₃ alkyl;
R³, R⁴=H;
Y¹=methoxy;
Y²=OH, methoxy; and
the α and β carbons are in the R configuration.
   Novel compounds of the present invention include those defined as follows:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹ and Y² are not OH;
R¹=C₁₋₃ alkyl;
R²=C₁₋₃ alkyl, Cl, Br, or I with the proviso that when X=OH, R² is not I or methyl; and
R³, R⁴=H, C₁₋₃ alkyl;
R⁵=C₁₋₆ alkyl; and
R⁶=H, C₁₋₆ alkyl.
   Preferred novel compounds are those wherein:
R¹=methyl;
R²=Br, C₁₋₃ alkyl; and
R³, R⁴=H.
   Most preferred novel compounds are those wherein:
R¹=methyl;
R²=Br, C₁₋₃ alkyl;
R³, R⁴=H;
Y¹=methoxy;
Y²=OH, methoxy; and
the α and β carbons are in the R configuration.

The present invention further relates to methods to lower and/or control normal or elevated intraocular pressure by administering an effective amount of a composition containing a compound having Formula I as described above.

The present invention also relates to a method for treating glaucoma which involves administering an effective amount of a composition containing a compound having Formula I as described above.

It is to be understood that both the foregoing general description and the following detailed description are exemplary and explanatory only and are intended to provide a further explanation of the present invention, as claimed.

### DETAILED DESCRIPTION OF THE PRESENT INVENTION

The present invention relates to a variety of compounds which are useful according to the present invention. These compounds are generally represented by the following Formula I. 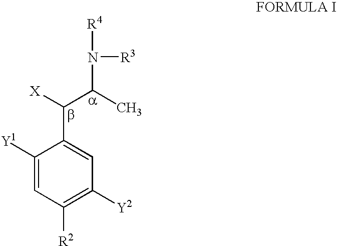
Wherein:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹ and Y² are not OH;
R¹=C₁₋₃ alkyl;
R²=C₁₋₃ alkyl, Cl, Br, I, CF₃, or OR¹;
R³, R⁴=H, C₁₋₃ alkyl;
R⁵=C₁₋₆ alkyl; and
R⁶=H, C₁₋₆ alkyl.
   Preferred compounds for lowering and maintaining IOP or treating glaucoma include compounds wherein:
R¹=methyl;
R²=Br, C₁₋₂ alkyl;
R³, R⁴=H;
Y¹=methoxy;
Y²=OH, methoxy; and
the α and β carbons are in the R configuration.
   Novel compounds of the present invention include those defined as follows:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹and Y² are not OH;
R¹=C₁₋₃ alkyl;
R²=C₁₋₃ alkyl, Cl, Br, or I with the proviso that when X=OH, R² is not I or methyl; and
R³, R⁴=H, C₁₋₃ alkyl;
R⁵=C₁₋₆ alkyl; and
R⁶=H, C₁₋₆ alkyl.
   Preferred novel compounds are those wherein:
R¹=methyl;
R²=Br, C₁₋₃ alkyl; and
R³, R⁴=H.
   Most preferred novel compounds are those wherein:
R¹=methyl;
R²=Br, C₁₋₃ alkyl;
R³, R⁴=H;
Y¹=methoxy;
Y²=OH, methoxy, and
the α and β carbons are in the R configuration.

Certain compounds of Formula I can contain one or more chiral centers. The present invention contemplates all enantiomers, diastereomers, and mixtures thereof, together with pharmaceutically acceptable salts thereof.

In the above definitions, the total number of carbon atoms in a substituent group is indicated by the Cᵢ₋ⱼ prefix where the numbers i and j define the number of carbon atoms. This definition includes straight chain, branched chain, and cyclic alkyl or (cyclic alkyl) alkyl groups.

In the formulas described above, the alkyl group can be straight-chain, branched or cyclic and the like.

The compounds of the present invention preferably function as 5-HT₂ agonists and preferably do not enter the CNS. Compounds having the ability to be a 5-HT₂ agonist are beneficial for controlling IOP as well as the treatment of glaucoma as shown in International Published Patent Application No. WO/16761, incorporated in its entirety by reference herein.

The compounds of the present invention preferably provide increased chemical stability and preferably achieve the desired level of therapeutic activity which includes a lowering or controlling of IOP.

The compounds of the present invention can be prepared using the techniques shown in the below set forth reaction schemes and Examples.

The compounds of the present invention can be used to lower and control IOP, including the IOP associated with normotension glaucoma, ocular hypertension, and glaucoma in mammals including humans. The compounds are preferably formulated in pharmaceutical compositions which are preferably suitable for topical delivery to the eye of the patient.

The compounds of this invention, Formula L can be incorporated into various types of ophthalmic formulations for delivery to the eye (e.g., topically, intracamerally, or via an implant). The compounds are preferably incorporated into topical ophthalmic formulations for delivery to the eye. The compounds may be combined with ophthalmologically acceptable preservatives, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity, such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-974, or the like, according to the published formulations for analogous ophthalmic preparations; preservatives and tonicity agents can be incorporated.

The compounds are preferably formulated as topical ophthalmic suspensions or solutions, with a pH of about 5 to 8. The compounds will normally be contained in these formulations in an amount 0.01% to 5% by weight, but preferably in an amount of 0.25% to 2% by weight. Thus, for topical presentation 1 to 2 drops of these formulations would be delivered to the surface of the eye 1 to 4 times per day according to the discretion of a skilled clinician.

The compounds can also be used in combination with other agents for lowering IPO and treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α1 antagonists (e.g., nipradolol), α2 agonists (e.g. iopidine and brimonidine), miotics (e.g., pilocarpine and epinephrine), prostaglandin analogs (e.g., latanoprost, travoprost, unoprostone, and compounds set forth in U.S. Pat. Nos. 5,889,052; 5,296,504; 5,422,368; and 5,151,444, “hypotensive lipids” (e.g., bimatoprost and compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203,350, and appropriate compounds from WO94/13275, including memantine. Such use in combination may be effected through concurrent or adjunctive administration, or through administration of a single composition comprising a combination of a compound of the present invention with one or more of the foregoing additional agents.

The following methods and examples are given to illustrate the preparation and effectiveness of compounds that are the subject of the present invention, but should not be construed as implying limitations to the claims.

### METHOD 1

### 5-HT₂ Receptor Binding Assay

To determine the affinities of serotonergic compounds at the 5-HT₂ receptors, their ability to compete for the binding of the agonist radioligand [¹²⁵I]DOI to brain 5-HT₂ receptors is determined as described below with minor modification of the literature procedure [Neuropharmacology, 26, 1803 (1987)]. Aliquots of post mortem rat cortex homogenates (400 μl) dispersed in 50 mM TrisHCl buffer (pH 7.4) are incubated with [¹²⁵I]DOI (80 pM final) in the absence or presence of methiothepin (10 μM final) to define total and non-specific binding, respectively, in a total volume of 0.5 ml. The assay mixture is incubated for 1 hour at 23° C. in polypropylene tubes and the assays terminated by rapid vacuum filtration over Whatman GF/B glass fiber filters previously soaked in 0.3% polyethyleneimine using ice-cold buffer. Test compounds (at different concentrations) are substituted for methiothepin. Filter-bound radioactivity is determined by scintillation spectrometry on a beta counter. The data are analyzed using a non-linear, iterative curve-fitting computer program [Trends Pharmacol. Sci., 16, 413 (1995)] to determine the compound affinity parameter. The concentration of the compound needed to inhibit the [¹²⁵ I]DOI binding by 50% of the maximum is termed the IC₅₀.

### METHOD 2

### 5-HT₂ Functional Assay: Phosphoinositide (PI) Turnover Assay

The relative agonist activity of serotonergic compounds at the 5-HT₂ receptor can be determined in vitro using the ability of the compounds to stimulate the production of [³H]inositol phosphates in [³H]myo-inositol-labeled A7r5 rat vascular smooth muscle cells by their ability to activate the enzyme phospholipase C. These cells are grown in culture plates, maintained in a humidified atmosphere of 5% CO₂ and 95% air and fed semi-weekly with Dulbecco's modified Eagle medium (DMEM) containing 4.5 g/L glucose and supplemented with 2 mM glutamine, 10 μg/ml gentamicin, and 10% fetal bovine serum. For the purpose of conducting the phosphoinositide (PI) turnover experiments, the A7r5 cells are cultured in 24-well plates as previously [J. Pharmacol. Expt. Ther. 286, 411 (1998)]. Confluent cells are exposed for 24-30 hrs to 1.5 μCi [³H]-myo-inositol (18.3 Ci/mmol) in 0.5 ml of serum-free medium. Cells are then rinsed once with DMEM/F-12 containing 10 mM LiCl prior to incubation with the test agent (or solvent as the control) in 1.0 mL of the same medium for 1 hr at 37° C., after which the medium is aspirated and 1 ml of cold 0.1 M formic acid added to stop the reaction. The chromatographic separation of [³H]-inositol phosphates ([³H]-IPs) on an AG-1-X8 column is performed as previously described [J. Pharmacol. Expt. Ther. 286, 411 (1998)] with sequential washes with H₂O and 50 mM ammonium formate, followed by elution of the total [³H]-IPs fraction with 1.2 M ammonium formate containing 0.1 M formic acid. The eluate (4 mL) is collected, mixed with 15 ml scintillation fluid, and the total [³H]-IPs determined by scintillation counting on a beta-counter. Concentration-response data are analyzed by the sigmoidal fit function of the Origin Scientific Graphics software (Microcal Software, Northampton, Mass.) to determine agonist potency (EC₅₀ value) and efficacy (Emax). Serotonin (5-HT) is used as a positive control (standard) agonist compound and the efficacy of test compounds is compared to that of 5-HT (set at 100%). The concentration of the compound needed to stimulate the production of [³H]-IPs by 50% of the maximum response is termed the EC₅₀ value.

### METHOD 3

### 5-HT₂ Functional Assay: [Ca²⁺]ᵢ Mobilization

The receptor-mediated mobilization on intracellular calcium ([Ca²⁺]ᵢ) was studied using the Fluorescence Imaging Plate Reader (FLIPR) instrument. Rat vascular smooth muscle cells, A7r5, were grown in a normal media of DMEM/10% FBS and 10 μg/mL gentamycin. Confluent cell monolayers were trypsinized, pelleted, and re-suspended in normal media. Cells were seeded in a 50 μL volume at a density of 20,000 cells/well in a black wall, 96-well tissue culture plate and grown for 2 days.

On the day of the experiment, one vial of FLIPR Calcium Assay Kit dye was re-suspended in 50 mL of a FLIPR buffer consisting of Hank's Balanced Salt Solution (HBSS), 20 mM HEPES, and 2.5 mM probenecid, pH 7.4. Cells were loaded with the calcium-sensitive dye by addition of an equal volume (50 μL) to each well of the 96-well plate and incubated with dye for 1 h at 23° C.

Typically, test compounds were stored at 25 μM in 50% DMSO/50% Ethanol solvent. Compounds were diluted 1:50 in 20% DMSO/20% Ethanol. For “hit” screening, compounds were further diluted 1:10 in FLIPR buffer and tested at a final concentration of 10 μM. For dose-response experiments, compounds were diluted 1:50 in FLIPR buffer and serially diluted 1:10 to give a 5- or 8-point dose-response curve.

The compound plate and cell plate were placed in the FLIPR instrument. At the beginning of an experimental run, a signal test was performed to check the basal fluorescence signal from the dye-loaded cells and the uniformity of the signal across the plate. The basal fluorescence was adjusted between 8000-12000 counts by modifying the exposure time, the camera F-stop, or the laser power. Instrument settings for a typical assay were the following: laser power 0.3-0.6 W, camera F-stop F/2, and exposure time 0.4 sec. An aliquot (25 μL) of the test compound was added to the existing 100 μL dye-loaded cells at a dispensing speed of 50 μL/sec. Fluorescence data were collected in real-time at 1.0 sec intervals for the first 60 secs and at 6.0 sec intervals for an additional 120 secs. Responses were measured as peak fluorescence intensity minus basal and where appropriate were expressed as a percentage of a maximum 5-HT-induced response.

The above procedures were used to generate the data shown in Table 1.

TABLE I

| | | 5-HT₂ | | | | |
|---|---|---|---|---|---|---|
| | | Binding | 5-HT_{2A} EC₅₀ | 5-HT_{2A} | 5-HT_{2A} | 5-HT_{2A} |
| | | (IC₅₀) nM | nM | % Emax | EC₅₀ nM | % Eₘₐₓ |
| Comp. | Structure | Common Name | (PI Assay) | (PI Assay) | (Ca⁺² Assay) | (Ca⁺² Assay) |
| 1 | 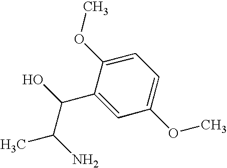 | >1,000 | | | | 9% |
| 2 | 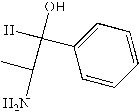 | 15,000 | | | | 1.2% |
| 3 | 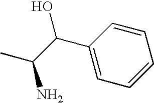 | >1,000 | | | | 1.9% |
| 4 | 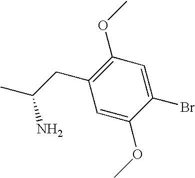 | 1 | | | 17.5 | 51.0% |
| 5 | 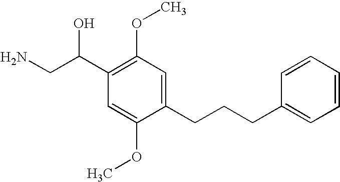 | 6.8 | | | | 3.1% |
| 6 | 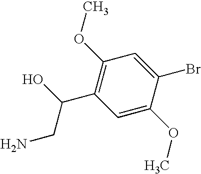 | 5.9 | | | | 7.8% |
| 7 | 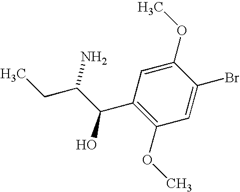 | 57 | | | | 4.8% |
| 8 | 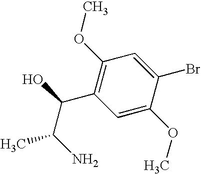 | 26 | 8000 | 57% | | 20% |
| 9 | 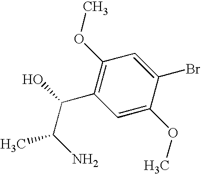 | 3.2 | 1180 | 68% | 84 | 22% |
| 10 | 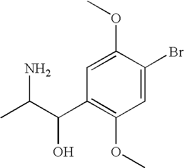 | 2.9 | | 47% | | 16% |
| 11 | 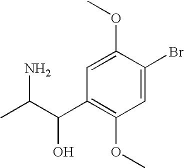 | 1.4 | 1130 | 102% | 96.9 | 54% |
| 12 | 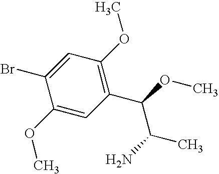 | 2.2 | 1080 | 100% | 1330 | 54% |
| 13 | 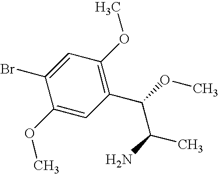 | 48.9 | 4610 | 117% | 5040 | 49% |
| 14 | 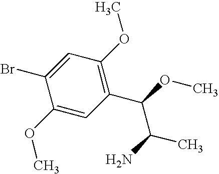 | 0.74 | 42.4 | 111% | 126 | 93% |
| 15 | 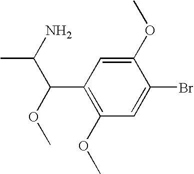 | 1.7 | | | 1410 | 31% |

Table 1 reports the 5-HT2 receptor affinity and function activity of a series of reference compounds (compounds 1-7) and examples of the compounds of this invention (8-15). Examples 8-15 have both high affinity for the 5-HT₂ receptor (IC₅₀<100 nM) and are functional agonists (% Eₘₐₓ>20%). The compounds of this invention are similar in potency to known 5-HT₂ agonist DOB (4).

### METHOD 4

### Intraocular Pressure Response in Lasered Monkeys

Intraocular pressure (IOP) was determined with an Alcon Pneumatonometer after light corneal anesthesia with 0.1% proparacaine. Eyes were washed with saline after each measurement. After a baseline IOP measurement, test compound was instilled in one 30 μL aliquot to the right eyes only of nine cynomolgus monkeys. Vehicle was instilled in the right eyes of six additional animals on the same schedule. IOP measurements were taken at 1, 3, and 6 hours after dosing.

Compound 9, a 5-HT₂ agonist, significantly lowered IOP in the lasered monkey eye by 10.7% (3.0 mmHg), 19% (7 mmHg) and 22.1% (8.1 mmHg) at 1, 3, and 6 hours, respectively in lasered monkeys after a single topical ocular instillation of 300 μg (Pharmacology Study No. 16744).

A single 300 μg topical ocular instillation compound 11 a serotonin 5-HT₂ agonist, lowered IOP in the lasered monkey eye by 19% (8 mmHg), 27.5% (11 mmHg), and 25.5% (10 mmHg) at 1, 3, and 6 hours, respectively (Pharmacology Study No. 16775).

### Synthesis of Compounds 9 and 8 from Table 1

Compound 9 and Compound 8 were prepared from Compound A and Compound B, respectively, which are identified and discussed below. The chiral purity of Compounds A and B were established by examination of the NMR spectra in the presence of the chiral shift reagent, Eu(hfbc) [McClure, D. E.; Arison, B. H.; Baldwin, J. J. Mode of nucleophilic addition of epichlorohydrin and related species: chiral aryloxymethyloxiranes. J. Am. Chem. Soc. 1979, 101, 3666-3668]. Chiral shift NMR analysis revealed none of the opposite enantiomer, indicating a chiral purity of>98 % for each isomer.

(S)-(−)-2-[N-(Trifluoroacetyl)amino]-1-(2,5-dimethoxy-4-bromophenyl)-1-propanone (Compound A). Oxalyl chloride (11.64 g, 91.8 mmol) was added in one portion to a stirred mixture of N-(trifluoroacetyl)-L-alanine [Weygand, F.; Leising, E. N-Trifluoracetylaminosäuren. II. Mitteil. Chem. Ber. 1954, 87, 248-256] (8.00 g, 43.2 mmol) and dry pyridine (0.5 mL) in dry CH₂Cl₂ (300 mL) at 0° C. under an N₂ atmosphere. The reaction mixture was allowed to warm to room temperature and to stir for an additional 2 h. The mixture was concentrated under reduced pressure at a temperature below 30° C. to give an oil which was mixed with 1-bromo-2,5-dimethoxybenzene (9.38 g, 43.2 mmol). The resulting mixture was dissolved in dry CH₂Cl₂ (25 mL) and added dropwise to a stirred solution of 1M TiCl₄ in CH₂Cl₂ (64.8 m L) at −50° C. under an N₂ atmosphere. The reaction mixture was allowed to warm to room temperature and to stir for an additional 60 h. After the reaction was complete, the reaction mixture was poured onto crushed ice. The organic portion was separated and washed successively with 1M HCl (2×50 ml), H₂O (2×50 mL), and saturated NaHCO₃ solution (2×50 mL). The solution was dried (MgSO₄) and evaporated to dryness under reduced pressure to give a crude brown product. The product was purified by flash chromatography (silica gel; CH₂Cl₂) and recrystallized from Et₂O/hexanes to yield 5.97 g (36%) of Compound A as a white solid: mp 144-145° C.; [a]_{D}=−28.9° (c 1, MeOH); ¹H NMR (CDCl₃) d 1.43 (d, J=6.2 Hz, 3H, CH₃), 3.90 (s, 3H, OCH₃), 3.95 (s, 3H, OCH₃), 5.59 (m, 1H, CH), 7.26 (s, 1H, ArH), 7.41 (s, 1H, ArH), 7.61. (bs, 1H, NHCO, exchangeable).

(R)-(+)-2-[N-(Trifluoroacetyl)amino]-1-(2,5-dimethoxy-4-bromophenyl)-1-propanone (Compound B). An exact replication of the above procedure using N-(trifluoroacetyl)-D-alanine [Fones, W. S. Some new N-acyl derivatives of alanine and phenylalanine. J. Org. Chem. 1952, 17, 1661-1665] gave 6.30 g (38%) of Compound B as a white crystals: mp 144-145° C.; [a]_{D}=+28.4° (c 1, MeOH).

Erythro isomers Compound 9 and Compound 8 were prepared by a highly erythro-selective reduction [Fujita, M.; Hiyama, T. Erythro-directive reduction of a-substituted alkanones by means of hydrosilanes in acidic media. J. Org. Chem. 1988, 53, 5415-5421] of the corresponding ketones Compound A and Compound B with dimethylphenylsilane in TFA.

(−)-erythro-(1R,2S)-1-Hydroxy-1-(4-bromo-2,5-dimethoxyphenyl)-2-aminopropane Hydro-chloride (Compound 9). Dimethylphenylsilane (1.70 g, 12.5 mmol) was added in dropwise manner to a solution of (S)-(−)-2-[N-(trifluoroacetyl)amino]-1-(2,5-dimethoxy-4-bromophenyl)-1-propanone (Compound A) (3.84 g, 10.0 mmol) in TFA (5 ml) at −5° C. under a N₂ atmosphere. The reaction mixture was allowed to warm to 0° C. and stirred for an additional 2 h. After the reaction was complete, the reaction mixture was poured onto crushed ice and neutralized with saturated NaHCO₃ solution. The solution was extracted with CH₂Cl₂ (3×50 mL). The combined CH₂Cl₂ portions were washed with saturated NaHCO₃ solution (3×25 mL), brine (3×25 mL), dried (MgSO₄) and evaporated to dryness under reduced pressure. The resulting residue was purified by flash chromatography with silica gel using, sequentially, CH₂Cl₂ and MeOH/CH₂Cl₂ (1:20) as eluants, and then dissolved in MeOH (30 ml). The solution was added to a stirred mixture of K₂CO₃ (6.91 g, 50 mmol) in H₂O (5 mL) and then heated at reflux for 2 h. MeOH was removed under reduced pressure and the residue was extracted with CH₂Cl₂ (3×25 mL). The combined organic portions were dried (MgSO₄), and the solvent was evaporated under reduced pressure to give the crude free base of Compound 9 as a white/yellowish solid. The free base was dissolved in anhydrous Et₂O (50 mL) and treated with ethereal HCl. The precipitated HCl salt was collected by filtration, washed with anhydrous Et₂O (2×10 mL), and recrystallized from EtOAc to afford 2.28 g (70%) of ALC-354 as a white crystals: mp 197-199° C.; [a]_{D}=−37.1° (c 1, MeOH); ¹H NMR (DMSO-d₆) d 0.92 (d, J=6.7 Hz, 3H, CH₃), 3.38 (m, 1H, CH—NH₃⁺), 3.76 (s, 3H, OCH₃), 3.79 (s, 3H, OCH₃), 5.06 (m, 1H, CH—OH), 6.06 (d, J=3.3 Hz, 1H, OH, exchangeable), 7.14 (s, 1H, ArH), 7.23 (s, 1H, ArH), 8.04 (br.s, 3H, NH₃⁺, exchangeable). Anal. (C₁₁H₁₆BrNO₃×HCl) C, H, N.

(+)-erythro-(1S,2R)-1-Hydroxy-1-(4-bromo-2,5-dimethoxyphenyl)-2-aminopropane Hydrochloride (ALC-355) was prepared from (R)-(+)-2-[N-(Trifluoroacetyl)amino]-1-(2,5-dimethoxy-4-bromophenyl)-1-propanone (Compound B) as a white crystals in 68% yield as described for Compound 9: mp 194-196° C.; [a]_{D}=+42.9° (c 1, MeOH); Anal. (C₁₁H₁₆BrNO₃×HCl×0.5H₂O) C, H, N.

### Synthesis of Compound 10 and Compound 11

Threo isomers Compounds 10 and 11 were prepared from the corresponding erythro compounds 9 and 8 using a modification of a procedure that was previously described for the preparation of threo norpseudoephedrines [Brauch, F.; Dralle, H.; Blanke, H. J. Ger. Offen. DE 3,408,850, Sep. 13, 1984; Chem. Abstr. 1985, 102, 24270p].

(+)-threo-(1S, 2S)-1-Hydroxy-1-(4-bromo-2,5-dimethoxyphenyl)-2-aminopropane Hydrochloride (Compound 10). Acetic anhydride (3.57 g, 35.0 mmol) was added to the free base of (−)-erythro-(1R,2S)-1-hydroxy-1-(4-bromo-2,5-dimethoxyphenyl)-2-aminopropane (2.90 g, 10.0 mmol) (Compound 9) at room temperature under a N₂ atmosphere. The reaction mixture was heated at 110° C. for 1 h and then cooled to 60-80° C. A solution of 60% aqueous H₂SO₄ (8 mL) was added and the reaction mixture was heated at 110° C. for an additional 1 h. The mixture was cooled to room temperature, poured onto crushed ice and basified with 15% aqueous NaOH solution until pH=8. The solution was extracted with CH₂Cl₂ (3×50 mL). The combined CH₂Cl₂ portions were washed with brine (3×50 mL), dried (MgSO₄) and evaporated under reduced pressure. The resulting residue was purified by flash chromatography (silica gel; CH₂Cl₂/MeOH (4:1)) to give an oil. The oil was dissolved in anhydrous Et₂O (50 mL) and treated with ethereal HCl. The precipitated HCl salt was collected by filtration, washed with anhydrous Et₂O (2×10 mL), and then recrystallized from Et₂O/MeOH to afford 2.67 g (82%) of Compound 10 as white crystals: mp 213-214° C.; [a]_{D}=+30.9° (c 1, MeOH); ¹H NMR (DMSO-d₆) d 1.03 (d, J=6.7 Hz, 3H, CH₃), 3.27 (m, 1H, CH—NH₃⁺), 3.76 (s, 3H, OCH₃), 3.79 (s, 3H, OCH₃), 4.84 (m, 1H, CH—OH), 6.16 (d, J=3.3 Hz, 1H, OH, exchangeable), 7.14 (s, 1H, ArH), 7.25 (s, 1H, ArH), 7.98 (br.s, 3H, NH₃⁺, exchangeable). Anal. (C₁₁H₁₆BrNO₃×HCl) C, H, N.

(−)-threo-(1R,2R)-1-Hydroxy-1-(4-bromo-2,5-dimethoxyphenyl)-2-aminopropane Hydrochloride (Compound 11) was prepared from (+)-erythro-(1S,2R)-1-hydroxy-1-(4-bromo-2,5-dimethoxyphenyl)-2-aminopropane (Compound 8) as white crystals in 80% yield as described for Compound 10: mp 214-215° C.; [a]_{D}=−31.3° (c 1, MeOH); Anal. (C₁₁H₁₆BrNO₃×HCl) C, H, N.

### Synthesis of Compound 12 and Compound 13

(−)-erythro-(1R,2S)-1-Methoxy-1-(4-bromo-2,5-dimethoxyphenyl)-2-aminopropane Oxalate (Compound 12). A solution of (−)-erythro-(1R,2S)-1-hydroxy-1-(4-bromo-2,5-dimethoxyphenyl)-2-aminopropane (free base of Compound 9) (2.90 g, 10.0 mmol) in THF (10 mL) was added in a dropwise manner to a suspension of 95% NaH (0.38 g, 15.0 mmol) in THF (5 mL) at 0° C. under a N₂ atmosphere. After stirring at room temperature for 0.5 h, the reaction mixture was treated in a dropwise manner with CH₃I (1.42 g, 10.0 mmol) at 0° C. and then heated at reflux for 1 h. The mixture was allowed to cool to room temperature, and then MeOH (3 mL) was added to destroy any excess NaH. The solution was concentrated under reduced pressure and diluted with H₂O (10 mL). The resulting mixture was extracted with CH₂Cl₂ (3×25 mL). The combined CH₂Cl₂ portions were washed with brine (3×25 mL), dried (MgSO₄) and evaporated under reduced pressure to give a crude oil. The oil was purified by flash chromatography (silica gel; CH₂Cl₂/MeOH, 9:1), dissolved in anhydrous Et₂O (50 mL), and treated with ethereal oxalic acid. The precipitated oxalate salt was collected by filtration, washed with anhydrous Et₂O (2×10 mL), and recrystallized from Et₂O/MeOH to afford 2.88 g (73%) of Compound 12 as a white crystals: mp 186-188° C.; [a]_{D}=−59.8° (c 1, MeOH); ¹H NMR (DMSO-d₆) d 0.95 (d, J=6.8 Hz, 3H, CH₃), 3.27 (s, 3H, CH—OCH₃) 3.40 (m, 1H, CH—NH₃⁺), 3.78 (s, 3H, OCH₃), 3.81 (s, 3H, OCH₃), 4.75 (d, J=2.8 Hz, 1H, CH—OCH₃), 6.91 (s, 1H, ArH), 7.30 (s, 1H, ArH). Anal. (C₁₂H₁₈BrNO₃×C₂H₂O₄) C, H, N.

(+)-erythro-(1S,2R)-1-Methoxy-1-(4-bromo-2,5-dimethoxyphenyl)-2-aminopropane Oxalate (Compound 13) was prepared from (+)-erythro-(1S,2R)-1-hydroxy-1-(4-bromo-2,5-dimethoxyphenyl)-2-aminopropane (free base of Compound 8) as a white crystals in 67% yield as described for Compound 12: mp 189-192° C.; [a]_{D}=+58.2° (c 1, MeOH); Anal. (C₁₁H₁₆BrNO₃×C₂H₂O₄) C, H, N.

### Synthesis of Compound 15 and Compound 14

(+)threo-(1S,2S)-1-Methoxy-1-(4-bromo-2,5-dimethoxyphenyl)-2-aminopropane Oxalate (Compound 15) was prepared from (+)-threo-(1S, 2S)-1-hydroxy-1-(4-bromo-2,5-dimethoxyphenyl)-2-aminopropane (Compound 10) as a white crystals in 52% yield as described for ALC-361: mp 115-118° C.; [a]_{D}=+51.7° (c 1, MeOH); ¹H NMR (DMSO-d₆) d 0.96 (d, J=6.7 Hz, 3H, CH₃), 3.14 (s, 3H, CH—OCH₃) 3.40 (m, 1H, CH—NH₃⁺), 3.78 (s, 3H, OCH₃), 3.81 (s, 3H, OCH₃), 4.55 (d, J=8.7 Hz, 1H, CH—OCH₃), 6.96 (s, 1H, ArH), 7.32 (s, 1H, ArH). Anal. (C₁₁H₁₆BrNO₃×C₂H₂O₄) C, H, N.

(−)-thero-(1R,2R)-1-Methoxy-1-(4-bromo-2,5-dimethoxyphenyl)-2-aminopropane Oxalate (Compound 14) was prepared from (−)-threo-(1R,2R)-1-hydroxy-1-(4-bromo-2,5-dimethoxyphenyl)-2-aminopropane (Compound 11) as a white crystals in 73% yield as described for Compound 12: mp 115-118° C.; [a]_{D}=−52.2° (c 1, MeOH); Anal. (C₁₁H₁₆BrNO₃×C₂H₂O₄) C, H, N.

### Synthesis of Compound 5

(±)1-Hydroxy-1-[4-(3-phenylpropyl)-2,5-dimethoxyphenyl]-2-aminoethane Hydrochloride (Compound 5). SnCl₄ (3.25 g, 12.5 mmol) was added in a dropwise manner to a solution of 1,4-dimethoxy-2-(3-phenylpropyl)benzene [Asano, M.; Aihara, T.; Aiko, I., Hasegawa, H. Syntheses of aryl- and aralkyl dihydroxybenzoquinones. Yakugaku Zasshi 1943, 63, 686-690; Chem. Abstr. 1952, 46, 93i] (2.56 g, 10.0 mmol) and Cl₂CHOCH₃ (1.15 g, 10.0 mmol) in CH₂Cl₂ (25 mL) at −10° C. under an N₂ atmosphere. After the addition was complete, the reaction mixture was allowed to warm to room temperature and stirred for an additional 2 h. The mixture was poured onto crushed ice. The organic portion was separated and washed with H₂O (2×100 mL), saturated NaHCO₃ solution (2×100 mL) and again with H₂O (2×100 mL). The solution was dried (MgSO₄) and evaporated under reduced pressure to give a yellow oil. The oil was dissolved in Et₂O (8 mL) and treated with saturated NaHSO₃ solution (50 mL). The resulting mixture was vigorously stirred for 12 h. The white precipitate was collected by filtration and washed with Et₂O (3×25 mL). The solid was suspended in saturated Na₂CO₃ solution (50 mL) and allowed to stir for 1 h. The mixture was extracted with CH₂Cl₂ (3×75 mL). The combined CH₂Cl₂ portions were washed with H₂O (3×50 mL), dried (MgSO₄), and evaporated under reduced pressure to give 2.55 g (90%) of 1-hydroxy-1-[4-(3-phenylpropyl)-2,5-dimethoxyphenyl]benzaldehyde as yellowish oil: ¹H NMR (CDCl₃) d 1.95 (m, 2H, CH₂), 2.68 (m, 4H, CH₂), 3.83 (s, 3H, OCH₃), 3.88 (s, 3H, OCH₃), 6.78 (s, 2H, ArH), 7.27 (m, 5H, ArH), 10.41 (s, 1H, CHO).

Nirtomethane (0.61 g, 10.0 mmol) was added in a dropwise manner to a solution of 1-hydroxy-1-[4-(3-phenylpropyl)-2,5-dimethoxyphenyl]benzaldehyde (2.84 g, 10.0 mmol) and CH₃ONa (0.67 g, 12.5 mmol) in MeOH (5 mL) at 0° C. under an N₂ atmosphere. After stirring at 0-5° C. for 2 h, the reaction mixture was treated with Et₂O (50 mL). The yellowish precipitate was collected by filtration and suspended in Et₂O (50 mL). Glacial AcOH (0.75 g, 12.5 mmol) was added and the white precipitate was removed by filtration. The filtrate was washed with H₂O (3×50 mL), dried (MgSO₄), and evaporated to dryness under reduced pressure to give 2.59 g (75%) of the crude 1-hydroxy-1-[4-(3-phenylpropyl)-2,5-dimethoxyphenyl]-2-nitroethane as a pale yellow solid. The product was used in the next step without any additional purification and characterization. PtO₂ (0.10 g, 0.4 mmol) was added to a solution of the solid (2.59 g, 7.5 mmol) in MeOH (50 mL) in a Parr bottle. This mixture was shaken at 50 psig of H₂ for 48 h. The catalyst was removed by filtration through a Celite pad and the filtrate was evaporated under reduced pressure to give a crude product. Purification by flash chromatography (silica gel; CH₂Cl₂/MeOH, 4:1) afforded the pure free base of Compound 5 as a white solid: mp 111-112° C. The solid was dissolved in anhydrous Et₂O (80 mL) and treated with ethereal HCl. The precipitated hydrochloride salt was collected by filtration, washed with anhydrous Et₂O (2×10 mL), and recrystallized from Et₂O/MeOH to afford 1.69 g (64%) of Compound 5 as a white crystals: mp 174-176° C.; ¹H NMR (DMSO-d₆) d 1.83 (m, 2H, CH₂), 2.58 (m, 4H, CH₂), 2.73 (m, 1H, CH₂), 2.96 (m, 1H, CH₂), 3.73 (s, 3H, OCH₃), 3.75 (s, 3H, OCH₃) 5.05 (m, 1H, CH—OH), 5.89 (d, J=4.1 Hz, 1H, OH, exchangeable), 6.81 (s, 1H, ArH), 7.04. (s, 1H, ArH),), 7.24 (s, 5H, ArH), 7.96 (br.s, 3H, NH₃⁺, exchangeable). Anal. (C₁₉H₂₅NO₃×HCl) C, H, N.

### Synthesis of Compound 7

(−)-erythro-(1R,2S)-1-Hydroxy-1-(4-bromo-2,5-dimethoxyphenyl)-2-aminobutane Oxalate (Compound 7). (S)-(−)-2-[N-(Trifluoroacetyl)amino]-1-(2,5-dimethoxy-4-bromophenyl)-1-butanone (Compound C) was prepared in 29% yield from (S)-(+)-2-trifluoroacetylaminobutyric acid [Fones, W. S.; Lee, M. Hydrolysis of the N-trifluroacetyl derivatives of several D- and L-amino acids by acylase I. J. Biol. Chem. 1954, 210, 227-238] exactly as described for the synthesis of Compound A. The product was isolated as a yellow/white powder: mp 92-94° C.; [a]_{D}=−5.7° (c 1, MeOH); ¹H NMR (CDCl₃) d 0.87 (t, J=7.6 Hz, 3H, CH₃), 1.61 (m, 2H, CH₂), 3.93 (s, 3H, OCH₃), 3.97 (s, 3H, OCH₃), 5.58 (m, 1H, CH), 7.28 (s, 1H, ArH), 7.41 (s, 1H, ArH), 7.44 (bs, 1H, NHCO, exchangeable). Using this as starting material, Compound 7 was prepared in the same manner described for the synthesis of Compound 9, except that ethereal oxalic acid was used to isolate the product as the oxalate salt. The salt was recrystallized from MeOH/Et₂O to afford ALC-391 as a white crystals in 76% yield: mp 203-205° C.; [a]_{D}=−28.5° (c 1, MeOH); ¹H NMR (DMSO-d₆) d 0.78 (t, J=7.3 Hz, 3H, CH₃), 1.33 (m, 2H, CH₂), 3.19 (m, 1H, CH—NH₃⁺), 3.77 (s, 3H, OCH₃), 3.80 (s, 3H, OCH₃), 5.10 (m, 1H, CH—OH), 7.17 (s, 1H, ArH), 7.23 (s, 1H, ArH). Anal. (C₁₂H₁₈BrNO₃×C₂H₂O₄) C, H, N.

### EXAMPLES

The following topical ophthalmic formulations are useful according to the present invention administered 1-4 times per day according to the discretion of a skilled clinician.

### Example 1

| Ingredients | Amount (wt %) |
|---|---|
| Compound 9 | 1% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting |
| | pH to 7.3-7.4 |
| Purified water | q.s. to 100% |

### Example 2

| Ingredients | Amount (wt %) |
|---|---|
| Compound 11 | 0.6% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting |
| | pH to 7.3-7.4 |
| Purified water | q.s. to 100% |

### Example 3

| Ingredients | Amount (wt %) |
|---|---|
| Compound 9 | 0.6% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting |
| | pH to 7.3-7.4 |
| Purified water | q.s. to 100 |

### Example 4

| Ingredients | Amount (wt %) |
|---|---|
| Compound 11 | 0.7% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting |
| | pH to 7.3-7.4 |

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

We claim:

1. A method for lowering and controlling intraocular pressure and/or treating a mammal suffering from glaucoma, which comprises, administering to the mammal a pharmaceutically effective amount of a compound of the following formula I: 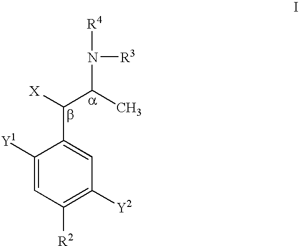
wherein:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹ and Y² are not OH;
R¹=C₁₋₃ alkyl;
R²=C₁₋₃ alkyl, Cl, Br, I, CF₃, or OR¹;
R³, R⁴=H, C₁₋₃ alkyl;
R⁵=C₁₋₆ alkyl; and
R⁶=H, C₁₋₆ alkyl;
and pharmaceutically acceptable salts thereof.

2. The method of claim 1, wherein for the compound of formula I:
R¹=methyl;
R²=Br, C₁₋₃ alkyl; and
R³, R⁴=H.

3. The method of claim 2, wherein for the compound of formula I;
Y¹=methoxy;
Y²=OH, methoxy; and
the α and β carbons are in the R configuration.

4. The method of claim 1, wherein the mammal is a human and the compound is administered topically.

5. The method of claim 1, which further comprises, administering an intraocular pressure (IOP) lowering effective amount of an IOP lowering agent selected from the group consisting of: β-blockers, carbonic anhydrase inhibitors, α2 agonists, prostaglandin analogs, and combinations thereof.

6. The method of claim 5, wherein the compound of formula I and the IOP lowering agent are administered together as a single composition.

7. The method of claim 1, wherein the compound of formula I is selected from the group consisting of: (−)-erythro-(1R,2S)-1-Hydroxy-1-(4-bromo-2,5-dimethoxyphenyl)-2-aminopropane Hydrochloride; (+)-erythro-(1S,2R)-1-Hydroxy-1-(4-bromo-2,5-dimethoxyphenyl)-2-aminopropane Hydrochloride; (+)-threo-(1S, 2S)-1-Hydroxy-1-(4-bromo-2,5-dimethoxyphenyl)-2-aminopropane Hydrochloride; (−)-threo-(1R,2R)-1-Hydroxy-1-(4-bromo-2,5-dimethoxyphenyl)-2-aminopropane Hydrochloride; (−)-erythro-(1R,2S)-1-Methoxy-1-(4-bromo-2,5-dimethoxyphenyl)-2-aminopropane Oxalate; (+)-erythro-(1S,2R)-1-Methoxy-1-(4-bromo-2,5-dimethoxyphenyl)-2-aminopropane Oxalate; (+)-threo-(1S,2S)-1-Methoxy-1-(4-bromo-2,5-dimethoxyphenyl)-2-aminopropane Oxalate; (−)-threo-(1R,2R)-1-Methoxy-1-(4-bromo-2,5-dimethoxyphenyl)-2-aminopropane Oxalate; and their pharmaceutically acceptable salts.

8. The method of claim 5, wherein the compound of formula I is: (−)-threo-(1R,2R)-1-Methoxy-1-(4-bromo-2,5-dimethoxyphenyl)-2-aminopropane Oxalate and its pharmaceutically acceptable salts.

9. A compound of the following formula I: 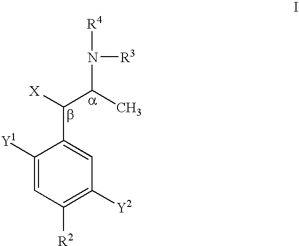
wherein:
X=OH, OR¹, OCON(R⁵, R⁶), or OCOR⁵;
Y¹=OH, OR¹, F, OCON(R⁵, R⁶), or OCOR⁵;
Y²=OH, OR¹, OCON(R⁵, R⁶), or OCOR⁵, with the proviso that both Y¹ and Y² are not OH;
R¹=C₁₋₃ alkyl;
R²=C₁₋₃ alkyl, Cl, Br, or I with the proviso that when X=OH, R² is not I or methyl;
R³, R⁴=H, C₁₋₃ alkyl;
R⁵=C₁₋₆ alkyl; and
R⁶=H, C₁₋₆ alky;
and pharmaceutically acceptable salts thereof.

10. The compound of claim 9, wherein for formula I:
R¹=methyl;
R²=Br, C₁₋₃ alkyl; and
R³, R⁴=H.

11. The compound of claim 10, wherein for formula I:
Y¹=methoxy;
Y²=OH, methoxy; and
the α and β carbons are in the R configuration.

12. The compound of claim 9, which is selected from the group consisting of: (−)-(erythro-(1R,2S)-1-Hydroxy-1-(4-bromo-2,5-dimethoxyphenyl)-2-aminopropane Hydrochloride; (+)-erythro-(1S,2R)-1-Hydroxy-1-(4-bromo-2,5dimethoxyphenyl)-2-aminopropane Hydrochloride; (+)-threo-(1S, 2S)-1-Hydroxy-1-(4-bromo-2,5-dimethoxyphenyl)-2-aminopropane Hydrochloride; (−)-threo-(1R,2R)-1-Hydroxy-1-(4-bromo-2,5-dimethoxyphenyl)-2-aminopropane Hydrochloride; (−)-erythro-(1R,2S)-1-Methoxy-1-(4-bromo-2,5-dimethoxyphenyl)-2-aminopropane Oxalate; (+)-erythro-(1S,2R)-1-Methoxy-1-(4-bromo-2,5-dimethoxyphenyl)-2-aminopropane Oxalate; (+)-threo-(1S,2S)-1-Methoxy-1-(4-bromo-2,5-dimethoxyphenyl)-2-aminopropane Oxalate; (−)-threo-(1R,2R)-1-Methoxy-1-(4-bromo-2,5-dimethoxyphenyl)-2-aminopropane Oxalate; and their pharmaceutically acceptable salts.

13. The compound of claim 12, which is:
(−)-threo-(1R,2R)-1-Methoxy-1-(4-bromo-2,5-dimethoxyphenyl)-2-aminopropane Oxalate.
